(19) United States
(12) Patent Application Publication
(10) Pub. No.: US 2006/0045911 A1
(43) Pub. Date: Mar. 2, 2006

(54) Stable pharmaceutical formulations

(75) Inventors: Nitin Bhalachandra Dharmadhikari, Mumbai (IN); Vaishali Vijay Dhavse, Mumbai (IN)

Correspondence Address: MERCHANT & GOULD PC, P.O. BOX 2903, MINNEAPOLIS, MN, 55402-0903, US

(73) Assignee: Sun Pharmaceutical Industries Ltd., Mumbai (IN)

(21) Appl. No.: 11/212,982

(22) Filed: Aug. 26, 2005

(30) Foreign Application Priority Data
Aug. 27, 2004 (IN) 927/MUM/2004

(51) Int. Cl. A61K 9/20; A61K 31/403

(52) U.S. Cl. 424/464; 514/412

(57) ABSTRACT

A stable oral pharmaceutical formulation comprising ramipril or its pharmaceutically acceptable salt and a stabilizing amount of an ammoniomethacrylate copolymer in a pharmaceutically acceptable carrier medium is described.

The present invention relates to stable oral pharmaceutical formulations comprising ramipril or its pharmaceutically acceptable salt and a stabilizing amount of an ammoniomethacrylate copolymer in a pharmaceutically acceptable carrier medium.

### BACKGROUND OF THE INVENTION

Ramipril is a 2-aza-bicyclo[3.3.0]-octane-3-carboxylic acid derivative with angiotensin-converting enzyme (ACE) inhibitor properties and has the following chemical name: (2S,3aS,6aS)-1[(S)—N—[(S)-1-Carboxy-3-phenylpropyl]alanyl]octahydrocyclopenta[b]pyrrole-2-carboxylic acid, 1-ethyl ester. Ramipril is converted to ramiprilat by hepatic cleavage of the ester group. Ramiprilat, the diacid metabolite of ramipril, is a non-sulfhydryl angiotensin converting enzyme inhibitor. Ramipril is indicated in reduction in risk of myocardial infarction, stroke, and death from cardiovascular causes, in hypertension and in heart failure post myocardial infarction. Ramipril is available in the United States of America as ALTACE hard shell capsules for oral administration and is available in strengths of 1.25 mg, 2.5 mg, 5 mg, and 10 mg. ACE inhibitors, such as ramipril, are generally very difficult to formulate into dosage forms because most ACE inhibitors undergo degradation upon contact with many of the excipients commonly used in pharmaceutical products, so that the product is not sufficiently stable to enable long shelf life. It is thus generally difficult to select the excipients that enable dosage forms with adequate stability. It has been found that ramipril shows a tendency to be unstable in pharmaceutical formulations, depending on the auxiliaries used, the manufacturing process and the storage. The main product of decomposition detected in pharmaceutical formulations is ramipril diketopiperazine (impurity D) produced by condensation and having the following chemical name: ethyl(2S)-2-[3S,5aS,8aS,9aS)-3-methyl-1,4-dioxodecahydro-2H-cyclopenta[4,5]pyrrolo[1,2-a]pyrazin-2-yl]-4phenylbutanone. This impurity is formed due to the degradation of ramipril under stress conditions of heating, acid addition, alkali addition and thermal oxidation.

ALTACE™ capsules contain ramipril in admixture with pregelatinised starch as the sole diluent, presumably because the manufacturer found pregelatinised starch to be the diluent that enabled the best stability. Although the stability of ALTACE™ capsules is sufficient to enable the capsules to be sold, the ramipril content does slowly degrade in ALTACE™ capsules, and it is desirable to enable solid dosage forms, and in particular capsules, with improved stability.

U.S. Pat. Nos. 5,151,433 and 5,442,008 (Hoechst Aktiengesellschaft) relate to method for stabilization of ramipril which comprises coating ramipril or its pharmaceutically acceptable salt, with a polymeric protective film, or comprises mixing ramipril or its pharmaceutically acceptable salt with a physiologically tolerated buffer which ensures that a pH in the weakly acid to weakly alkaline range is set up in a formulation in the presence of moisture, and ramipril which has been stabilized by a polymeric protective film or by mixture with a buffer. The pharmaceutical composition in compressed form, containing ramipril in the form of an agglomerate is stabilized with a polymeric protective coating, wherein the proportion by weight of the polymeric protective coating is 3 to 25% relative to ramipril. The patent teaches that decomposition of ramipril is favored by mechanical stress during formulating the dosage form as well as with increasing temperature and moisture that the formulation may be subjected to, during storage. The use of a protective coating of polymeric film-formers around the ramipril, counteracts the decomposition of ramipril due to mechanical stress. The invention of this patent uses a layer or coating of a polymer having a defined thickness to prevent damage to ramipril due to mechanical stress. The present invention uses a polymer intimately mixed with ramipril, in order to provide a microenvironment, which stabilizes the drug.

PCT Application number WO 03/028707A1 (Sherman, B; C) relates to a solid pharmaceutical composition for oral administration comprising ramipril and lactose monohydrate. It is disclosed that the use of lactose monohydrate as a diluent provides a ramipril formulation with stability superior to that achieved by using either anhydrous lactose or starch as diluent (similar to that used in ALTACE™ capsules).

PCT application number WO 03/059330A1 claims a stable pharmaceutical composition wherein a compressed core is coated with an ACE inhibitor, such as ramipril. This process avoids degradation of the ACE inhibitor to the diketopiperazine due to mechanical stress, and also avoids direct contact of the auxiliaries used in the composition with the ACE inhibitor thereby further preventing degradation. U.S. application No. 20030215526A1 claims a pharmaceutical composition comprising an ACE inhibitor susceptible to degradation, mixed with a greater than stoichiometric amount of an alkali or alkaline earth metal carbonate.

PCT application number WO 2005/067887A2 claims a stable tablet formulation comprising ramipril, calcium sulphate dihydrate and sodium hydrogen carbonate optionally in combination with a disintegrant, binder and lubricant and other excipients.

In view of the prior art, there lies a need to provide a simple method of preparing stable oral pharmaceutical formulations comprising ramipril or its pharmaceutically acceptable salt in a pharmaceutically acceptable carrier medium.

### OBJECT OF THE INVENTION

It is an object of the present invention to provide stable oral pharmaceutical formulations comprising ramipril or its pharmaceutically acceptable salt and an ammoniomethacrylate copolymer in a pharmaceutically acceptable carrier medium.

### SUMMARY OF THE INVENTION

The present invention provides stable oral pharmaceutical formulations comprising ramipril or its pharmaceutically acceptable salt and an ammoniomethacrylate copolymer in an amount sufficient to stabilize the formulation, in a pharmaceutically acceptable carrier medium.

### DETAILED DESCRIPTION OF THE INVENTION

The present invention provides stable oral pharmaceutical formulations comprising ramipril or its pharmaceutically acceptable salt and an ammoniomethacrylate copolymer in an amount sufficient to stabilize the formulation, in a pharmaceutically acceptable carrier medium.

The formulation of the present invention uses a stabilizer in order to provide a microenvironment that protects the ramipril from degradation. The stabilizer used is an ammoniomethacrylate copolymer. The preferred ammoniomethacrylate copolymer is the commercially available Eudragit RSPO. The ammoniomethacrylate copolymer may be used in amounts ranging from 10 to 100 mg. The ammoniomethacrylate polymer is used in an amount sufficient to provide a formulation that is stable, i.e. the impurity D during the shelf life of the formulation is less than 1%.

The excipients-compatibility studies carried out indicate that certain conventional excipients cause degradation of ramipril with corresponding high levels of impurities, specifically the impurity D. These conventional excipients include, but not limited to, lactose anhydrous, crospovidone, magnesium hydroxide, lactitol, hydroxypropyl cellulose, maltodextrin, magnesium aluminium silicate, talc, dicalcium phosphate anhydrous, powdered cellulose, Eudragit E100, microcrystalline cellulose, calcium carbonate, sodium carboxymethyl cellulose, meglumine, pregelatinized starch, magnesium stearate, dextrose monohydrate, sodium starch glycolate, calcium silicate, colloidal silicon dioxide, citric acid, sodium bicarbonate, stearic acid and the like. According to the present invention, the use of an ammoniomethacrylate polymer in sufficient amounts makes it possible to use pharmaceutical excipients conventionally used in the art to obtain oral stable pharmaceutical formulations comprising ramipril or its pharmaceutically acceptable salt with acceptable levels of impurity D.

The examples that follow are provided as illustrations and do not limit the scope of the present invention.

### EXAMPLE 1-4

The oral formulations of the present invention were obtained as per the procedure given in Table 1 below.

TABLE 1

| | | Quantity (mg/tablet) | | | |
|---|---|---|---|---|---|
| | | Example | Example | Example | Example |
| Sr. No. | Ingredients | 1 | 2 | 3 | 4 |
| 1. | Ramipril | 1.25 | 2.5 | 5.0 | 10.0 |
| 2. | Pregelatinised | 99.0 | 97.75 | 95.25 | 90.25 |
| | starch- | | | | |
| | Starch | | | | |
| | 1500 LM | | | | |
| 3. | Ammonio- | 24.75 | 24.75 | 24.75 | 24.75 |
| | methacrylate | | | | |
| | copolymer | | | | |
| | (Eudragit | | | | |
| | RSPO) | | | | |
| | Total | 125.0 | 125.0 | 125.0 | 125.0 |

Ramipril was blended with pregelatinised starch and Eudragit RSPO in geometric proportion in a double cone blender. This blend was filled into empty hard gelatin capsule shells (Size 3 or Size 4) at fill weight of 125 mg on hand capsule filling machine.

TABLE 2
Example 5-8 (comparative Examples)

| | | Quantity (mg/tablet) | | | |
|---|---|---|---|---|---|
| | | Example | Example | Example | Example |
| Sr. No. | Ingredients | 5 | 6 | 7 | 8 |
| 1. | Ramipril | 1.25 | 2.5 | 5.0 | 10.0 |
| 2. | Pregelatinised | 123.75 | 122.5 | 120.0 | 115.0 |
| | starch- | | | | |
| | Starch | | | | |
| | 1500 LM | | | | |
| | Total | 125.0 | 125.0 | 125.0 | 125.0 |

Ramipril was blended with pregelatinised starch in geometric proportion in a double cone blender. This blend was filled into empty hard gelatin capsule shells (Size 3 or Size 4) at fill weight of 125 mg on hand capsule filling machine.

The stability of the formulation of example 1-8 was carried out in HDPE bottles with screw on cap at 40° C./75% RH for 3 months and at 25° C./60% RH for 12 months. The comparative results for percentage Impurity D are given in table 3 and table 4 respectively.

TABLE 3

| | Initial | 3 month 40° C./75% R.H. |
|---|---|---|
| | Impurity D (% w/w) | Impurity D (% w/w) |
| Examples | | |
| 1 | 0.17 | 3.37 |
| 2 | 0.72 | 2.09 |
| 3 | 0.50 | 1.51 |
| 4 | 0.38 | 1.09 |
| Comparative Examples | | |
| 5 | 0.23 | 6.58 |
| 6 | 0.24 | 5.12 |
| 7 | 0.18 | 2.47 |
| 8 | 0.22 | 1.46 |

TABLE 4

| | Initial | 12 month 25° C./60% R.H. |
|---|---|---|
| | Impurity D (% w/w) | Impurity D (% w/w) |
| Examples | | |
| 1 | 0.17 | 2.23 |
| 2 | 0.72 | 1.38 |
| 3 | 0.50 | 0.85 |
| 4 | 0.38 | 0.35 |
| Comparative examples | | |
| 5 | 0.23 | 6.01 |
| 6 | 0.24 | 3.43 |
| 7 | 0.18 | 1.50 |
| 8 | 0.22 | 0.76 |

We claim:

1. A stable oral pharmaceutical formulation comprising ramipril or its pharmaceutically acceptable salt and a stabilizing amount of an ammoniomethacrylate copolymer in a pharmaceutically acceptable carrier medium.

2. A stable oral pharmaceutical formulation as claimed in claim 1 wherein the ammoniomethacrylate copolymer is Eudragit RSPO.

3. A stable oral pharmaceutical formulation as claimed in claim 1 wherein the pharmaceutically acceptable carrier medium comprises one or more fillers and lubricants.

4. A stable oral pharmaceutical formulation as claimed in claim 1 wherein a stabilizing amount of ammoniomethacrylate copolymer is intimately mixed with ramipril or its pharmaceutically acceptable salt.

5. A stable oral pharmaceutical formulation as claimed in claim 4 wherein the pharmaceutically acceptable carrier medium comprises one or more fillers and lubricants.
